(19) United States
(12) Patent Application Publication
(10) Pub. No.: US 2020/0275863 A1
(43) Pub. Date: Sep. 3, 2020

(54) Aging Measuring Instrument Based on Walking Speed

(71) Applicant: DYPHI INC., Daejeon (KR)

(72) Inventors: Hee Won Jung, Bucheon-si, Gyeonggi-do (KR); Hyun Chul Roh, Daejeon (KR)

(21) Appl. No.: 16/647,460

(22) PCT Filed: Sep. 6, 2018
(86) PCT No.: PCT/KR2018/010412

§ 371 Date: Mar. 13, 2020

(30) Foreign Application Priority Data
Sep. 14, 2017 (KR) 10-2017-0117752

(51) Int. Cl. A61B 5/11; A61B 5/00

(52) U.S. Cl. CPC A61B 5/112; A61B 2503/08; A61B 5/742; A61B 5/7275

(57) ABSTRACT

Provided is a frailty diagnosis method including: setting, by a processor, a setting value for measuring a walking speed according to positions of at least two measuring devices; detecting, by the at least two measuring devices, a movement of a frailty diagnosis-target person; measuring, by the processor, a walking speed of the frailty diagnosis-target person based on the movement of the frailty diagnosis-target person according to the setting value; and determining, by the processor, a frailty index of the frailty diagnosis-target person based on the measured walking speed.

### TECHNICAL FIELD

The present disclosure relates to a method and apparatus for measuring the frailty index of a frailty diagnosis-target person, and more particularly, to a method and apparatus for measuring the frailty index of a frailty diagnosis-target person based on the walking speed of the frailty diagnosis-target person.

### BACKGROUND ART

As the population ages, more older patients are undergoing medical care such as chemotherapy that may cause complications, and surgical operations of various severities ranging from minor outpatient surgery to major surgery inevitably requiring intensive postoperative treatment. In such various medical treatments, it is important to assess the physiological functions of patients for preventing complications and unnecessary treatments. However, previous studies have shown that the physiological reserve of patients is not properly predicted by the chronological ages of the patients. On the contrary, it has been known that the assessment of “frailty,” defined as physiological homeostasis deteriorated by aging, is more useful to predict complications, future dysfunctions, deaths, or the like caused by medical and surgical treatment than the assessment of chronological ages or classical risk assessment tools.

In general, such a classical frailty assessing method, comprehensive geriatric assessment (CGA), is performed by assessing details of an individual's comorbidity, medication status, activities of daily living (ADL), instrumental activities of daily living (IADL), cognitive function, depression, social support, physical functions, or the like. However, the comprehensive geriatric assessment requires professionally trained manpower and a long period of time, and thus, it is difficult to widely use the comprehensive geriatric assessment everywhere other than in specialized geriatric centers. Therefore, there has been an increasing need for a method of quickly and objectively screening frailty during busy outpatient care in various specialized areas for treating older adults.

### DESCRIPTION OF EMBODIMENTS

### Technical Problem

The present disclosure provides a frailty diagnosis method of measuring the frailty index of a frailty diagnosis-target person based on the walking speed of the frailty diagnosis-target person. In addition, the present disclosure provides a frailty diagnosis apparatus for performing the frailty diagnosis method. In addition, the present disclosure provides a non-transitory storage medium having recorded thereon a program for executing the frailty diagnosis method.

### Solution to Problem

Provided is a frailty diagnosis method including: setting, by a processor, a setting value for measuring a walking speed in a given measuring environment according to positions of at least two measuring devices; detecting, by the at least two measuring devices, a movement of a frailty diagnosis-target person; measuring, by the processor, a walking speed of the frailty diagnosis-target person based on the movement of the frailty diagnosis-target person according to the setting value; and determining, by the processor, a frailty index of the frailty diagnosis-target person based on the measured walking speed.

Provided is a frailty diagnosis apparatus including: at least two measuring devices configured to detect a movement of a frailty diagnosis-target person; and a processor configured to set a setting value for measuring a walking speed according to positions of the at least two measuring devices, measure a walking speed of the frailty diagnosis-target person based on the movement of the frailty diagnosis-target person according to the setting value, and determine a frailty index of the frailty diagnosis-target person based on the measured walking speed.

Provided is a non-transitory computer-readable recording medium having recorded thereon a program for executing the frailty diagnosis method according to various embodiments of the present disclosure.

### Advantageous Effects of Disclosure

According to the frailty diagnosis method and apparatus of the present disclosure, the frailty index of a frailty diagnosis-target person may be rapidly and objectively determined.

### BRIEF DESCRIPTION OF DRAWINGS

FIG. 1 illustrates an embodiment of a frailty diagnosis apparatus for determining the frailty index of a frailty diagnosis-target person based on the walking speed of the frailty diagnosis-target person.

FIG. 2 illustrates an embodiment of a frailty diagnosis method for determining the frailty index of a frailty diagnosis-target person based on the walking speed of the frailty diagnosis-target person.

FIGS. 3A and 3B are two-dimensional graphs respectively illustrating a relationship between actual age and walking speed and a relationship between frailty index and walking speed.

FIG. 4 is a graph illustrating the survival probabilities of groups having certain walking speeds by Kaplan-Meier analysis.

FIG. 5 illustrates distributions of the walking speed of older adults living in a community in Korea according to the genders.

FIGS. 6A to 6C are graphs for estimating a frailty index by using a walking speed and other measured values.

In the following descriptions of the accompanying drawings, like reference numbers denote like elements unless otherwise specified, and overlapping descriptions thereof are not provided.

### BEST MODE

Provided is a frailty diagnosis method including: setting, by a processor, a setting value for measuring a walking speed in a given measuring environment according to positions of at least two measuring devices: detecting, by the at least two measuring devices, a movement of a frailty diagnosis-target person; measuring, by the processor, a walking speed of the frailty diagnosis-target person based on the movement of the frailty diagnosis-target person according to the setting value; and determining, by the processor, a frailty index of the frailty diagnosis-target person based on the measured walking speed.

The setting of the setting value may be performed according to relative positions of the at least two measuring devices.

The setting of the setting value may be periodically performed according to a preset period.

The setting of the setting value may be performed when a variation in the positions of the at least two measuring devices is detected.

The at least two measuring devices may include a first measuring device and a second measuring device. The detecting of the movement of the frailty diagnosis-target person may include: detecting, by the first measuring device, a movement of the frailty diagnosis-target person, and recording, by the first measuring device, at least one of a first detection time and a first detection distance of the frailty diagnosis-target person; and detecting, by the second measuring device, a movement of the frailty diagnosis-target person, and recording, by the second measuring device, at least one of a second detection time and a second detection distance of the frailty diagnosis-target person.

The measuring of the walking speed of the frailty diagnosis-target person may be performed by calculating the walking speed of the frailty diagnosis-target person according to the first detection time and the second detection time.

The determining of the frailty index of the frailty diagnosis-target person may include: determining, by the processor, a walking speed parameter representing the walking speed; and determining, by the processor, the frailty index such that the frailty index corresponds to the walking speed parameter. The walking speed parameter may represent a walking speed in a specific section.

The determining of the frailty index of the frailty diagnosis-target person may be performed by determining the frailty index based on the walking speed according to a walking speed-frailty index correlation function, which indicates a relationship between the walking speed and the frailty index.

The determining of the frailty index of the frailty diagnosis-target person may include: obtaining frailty parameters of the frailty diagnosis-target person in addition to a walking speed parameter of the frailty diagnosis-target person; and determining the frailty index of the frailty diagnosis-target person based on the walking speed parameter and at least one of the frailty parameters, wherein the frailty parameters may include a muscle decrease parameter relating to a muscle function of the frailty diagnosis-target person and a motor ability parameter relating to motor ability of the frailty diagnosis-target person.

The frailty diagnosis method may further include inferring, by the processor, a health state of the frailty diagnosis-target person based on the frailty index, age, and gender of the frailty diagnosis-target person.

The frailty diagnosis method may further include displaying, on a display, at least one of the walking speed and the frailty index.

Provided is a non-transitory computer-system-readable storage medium having recorded thereon a program for executing the frailty diagnosis method.

Provided is a frailty diagnosis apparatus including: at least two measuring devices configured to detect a movement of a frailty diagnosis-target person; and a processor configured to set a setting value for measuring a walking speed in a given measuring environment according to positions of the at least two measuring devices, measure a walking speed of the frailty diagnosis-target person based on the movement of the frailty diagnosis-target person according to the setting value, and determine a frailty index of the frailty diagnosis-target person based on the measured walking speed.

### MODE OF DISCLOSURE

Advantages and features of embodiments, and implementation methods thereof will be clarified through the following descriptions given with reference to the accompanying drawings. However, the present disclosure is not limited to the embodiments disclosed below, but may be embodied in various different forms. Rather, these embodiments are provided so that this disclosure will be thorough and complete, and will fully convey the scope of the present disclosure to those of ordinary skill in the art.

Terms used herein will be briefly described below, and embodiments will be described in detail.

In the present specification, the term “frailty index” refers to an index indicating the degree of facility of older adults. The frailty index of an individual is expressed by the ratio of the number of frailty-related symptoms that the individual has to the total number of frailty-related symptoms. Therefore, when an individual has no frailty-related symptoms, the frailty index of the individual is 0, and on the contrary, when an individual has all the frailty-related symptoms, the frailty index of the individual is 1. In other words, when an older person has a high frailty index, the individual has a severe degree of frailty. The number and types of symptoms used to measure the frailty index may be adjusted by a measurer.

In the present specification, the term “cohort” refers to a group who shares a certain statistical factor. Cohort studies may be conducted to track the incidence of a disease of concern depending on a particular factor by comparing a group exposed to the particular factor with a group not exposed to the particular factor. For example, cohort studies may be conducted to track the survival rates of groups classified according to the walking speeds or frailty indexes thereof.

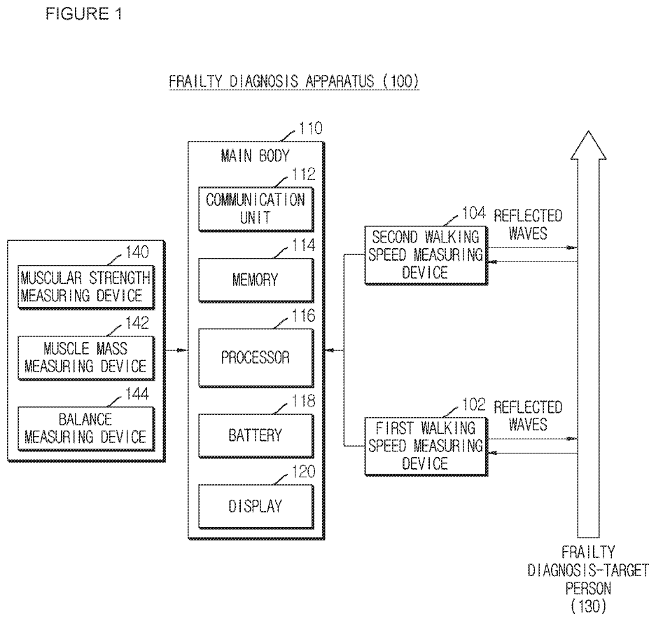

FIG. 1 illustrates an embodiment of a frailty diagnosis apparatus 100 for determining the frailty index of a frailty diagnosis-target person 130 based on the walking speed of the frailty diagnosis-target person 130.

The frailty diagnosis apparatus 100 may include a first walking speed measuring device 102, a second walking speed measuring device 104, and a main body 110. The main body 110 may include a communication unit 112, a memory 114, a processor 116, a battery 118, and a display 120. In other embodiments, the frailty diagnosis apparatus 100 may further include additional elements necessary for diagnosing frailty.

The first walking speed measuring device 102 may be separate from the second walking speed measuring device 104.

The first walking speed measuring device 102 and the second walking speed measuring device 104 detect the movement of the frailty diagnosis-target person 130. The first walking speed measuring device 102 and the second walking speed measuring device 104 may emit laser light or ultrasonic waves and may recognize reflected laser light or ultrasonic waves. In addition, the first walking speed measuring device 102 and the second walking speed measuring device 104 may detect the movement of the frailty diagnosis-target person 130 based on a recognized laser or ultrasonic signal.

When the movement of the frailty diagnosis-target person 130 is detected, the first walking speed measuring device 102 and the second walking speed measuring device 104 may transmit detection times to the communication unit 112. In addition, the first walking speed measuring device 102 and the second walking speed measuring device 104 may transmit the distances from the first walking speed measuring device 102 and the second walking speed measuring device 104 to the frailty diagnosis-target person 130 to the communication unit 112 or the processor 116.

The first walking speed measuring device 102 and the second walking speed measuring device 104 periodically check the relative distance therebetween. In addition, the first walking speed measuring device 102 and the second walking speed measuring device 104 may periodically transmit the relative distance to the communication unit 112 or the processor 116.

Information transmitted from the first walking speed measuring device 102 and the second walking speed measuring device 104 to the communication unit 112 or the processor 116 may be distinguished using separate identifiers, Information transmitted to the communication unit 112 may be stored in the memory 114 or may be processed by the processor 116.

The first walking speed measuring device 102 or the second walking speed measuring device 104 may encode and modulate information necessary to derive the speed of walking and may transmit the encoded and modulated information to the communication unit 112.

In FIG. 1, two walking speed measuring devices 102 and 104 are illustrated. However, in other embodiments, an extra walking speed measuring device may be additionally arranged.

In FIG. 1, the main body 110 is physically separate from the first walking speed measuring device 102 and the second walking speed measuring device 104. However, the main body 110 may be physically coupled with the first walking speed measuring device 102 or the second walking speed measuring device 104.

The main body 110 may include the communication unit 112 to receive information transmitted from the first walking speed measuring device 102 or the second walking speed measuring device 104. The communication unit 112 may demodulate and decode a signal transmitted from the first walking speed measuring device 102 or the second walking speed measuring device 104 to recover information that the first walking speed measuring device 102 or the second walking speed measuring device 104 has obtained.

The main body 110 may include the memory 114 to store information obtained through the communication unit 112 or store information processed by the processor 116. In addition, the memory 114 may store information necessary for information-processing processes of the processor 116, such as information about walking speed parameters, information about a correlation between walking speed and frailty index, and information about combinations of walking speeds and other measured values for determining frailty.

The main body 110 may include the processor 116 to determine the frailty index of the frailty diagnosis-target person 130 based on information transmitted from the first walking speed measuring device 102 or the second walking speed measuring device 104.

The processor 116 may set setting values for measuring a walking speed according to the position of the first walking speed measuring device 102 or the second walking speed measuring device 104. For example, the processor 116 may calculate the relative distance between the first walking speed measuring device 102 and the second walking speed measuring device 104 according to the position of the first walking speed measuring device 102 and the position of the second walking speed measuring device 104.

According to an embodiment, the processor 116 may periodically set the setting values according to a setting cycle. According to another embodiment, the processor 116 may set the setting values when the position of at least one of the first walking speed measuring device 102 and the second walking speed measuring device 104 is changed. The processor 116 may set the setting values periodically or when a position change is detected, so as to reflect changes in the positions of the first walking speed measuring device 102 and the second walking speed measuring device 104.

The processor 116 may calculate the walking speed of the frailty diagnosis-target person 130 based on at least one of the setting values. For example, the processor 116 may calculate the speed of walking based on a set relative distance. Specifically, the processor 116 may calculate the walking speed of the frailty diagnosis-target person 130 by dividing the set relative distance by the difference between a detection time of the first walking speed measuring device 102 and a detection time of the second walking speed measuring device 104.

The processor 116 may adjust the relative distance between the first walking speed measuring device 102 and the second walking speed measuring device 104 by considering the distances from the frailty diagnosis-target person 130 to the first walking speed measuring device 102 and the second walking speed measuring device 104. Due to the difference between the measurement angle of the first walking speed measuring device 102 and the measurement angle of the second walking speed measuring device 104, the actual moving distance of the frailty diagnosis-target person 130 may be distorted. Therefore, the first walking speed measuring device 102 and the second walking speed measuring device 104 may be installed in parallel, and the actual moving distance of the frailty diagnosis-target person 130 may be accurately calculated by adjusting the relative distance between the first walking speed measuring device 102 and the second walking speed measuring device 104.

The processor 116 may determine the frailty index of the frailty diagnosis-target person 130 based on the walking speed of the frailty diagnosis-target person 130. The speed of walking is an index commonly used to determine a frailty index. Therefore, the processor 116 may calculate the physiological age of the frailty diagnosis-target person 130 according to the walking speed of the frailty diagnosis-target person 130 with reference to a correlation between walking speed and frailty index.

The processor 116 may determine a walking speed parameter representing the speed of walking. The walking speed parameter represents a walking speed in a specific section. For example, the walking speed parameter may be defined for each section having a size of 0.2 m/s. In a specific example, the walking speed parameter may be defined as 1 for a section of 0.4 m/s to 0.6 m/s and as 2 for a section of 0.6 m/s to 0.8 m/s. In addition, the walking speed parameter may be defined for the rest sections each having a size of 0.2 m/s. The above example is merely an illustrative example, and values of the walking speed parameter and sections corresponding thereto may be easily changed by those of ordinary skill in the art.

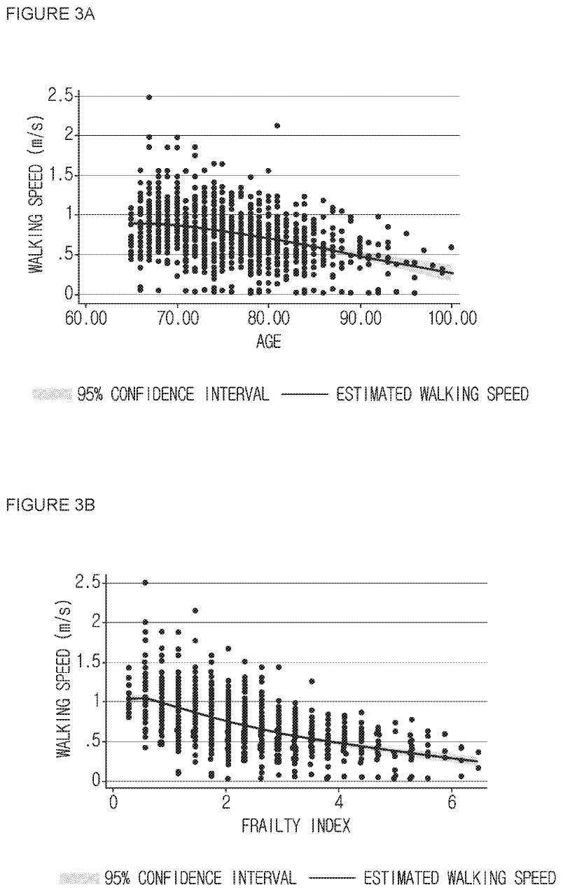

The processor 116 may determine a frailty index based on the speed of walking with reference to a walking speed-frailty index correlation indicating a relationship between walking speed and frailty index. When the speed of walking is expressed by the walking speed parameter, the processor 116 may determine a frailty index based on the walking speed parameter with reference to the walking speed-frailty index correlation. The walking speed-frailty index correlation may be determined by regression analysis of statistical data about walking speeds and frailty indexes. The walking speed-frailty index correlation will be specifically explained in the description of FIG. 3.

The processor 116 may derive the health state of the frailty diagnosis-target person 130 based on the physiological age of the frailty diagnosis-target person 130 determined based on the frailty index of the frailty diagnosis-target person 130, the actual age and gender of the frailty diagnosis-target person 130, etc. The processor 116 may calculate information such as postoperative mortality and postoperative complication rates according to information such as the frailty index. Therefore, the processor 116 may help users of the frailty diagnosis apparatus 100 in determining treatment methods for the frailty diagnosis-target person 130.

The processor 116 may determine a frailty index by using not only the speed of walking but also other measured values. In addition to the speed of walking, other factors may be additionally used to determine a frailty index. For example, data obtained through muscle strength assessment, muscle mass assessment, balance assessment, or the like may be additionally considered.

To obtain such additional data, additional measuring devices 140, 142, and 144 may be installed separately from the first and second walking speed measuring devices 102 and 104. For example, a muscular strength measuring device 140 may be installed for muscle strength assessment, a muscle mass measuring device 142 may be installed for muscle mass assessment, and a balance measuring device 144 may be installed for balance assessment.

The processor 116 may perform such calculations based on statistical data. Examples of the statistical data include statistical data about the above-mentioned walking speed-frailty index correlation, and statistical data about the walking speed, actual age, and gender of the frailty diagnosis-target person 130. In addition, statistical data on other factors related to the frailty index may also be used.

The statistical data is stored in the memory 114 or a memory (not shown) provided outside the main body 110. The processor 116 may periodically update the statistical data.

Two or more processors 116 may be provided, and when a plurality of processors 116 are used, the processors 116 do not necessarily need to be located at physically adjacent distances from each other.

The main body 110 may include the battery 118 that stores electrical energy for smooth operations of the elements of the main body 110. The battery 118 may include a charger that receives electrical energy from the outside of the main body 110. In addition, the battery 118 may include a voltage regulator to supply an appropriate voltage to the elements of the main body 110.

The main body 110 may include the display 120, which displays results of calculation of the processor 116. For example, the display 120 may display values measured or calculated by the frailty diagnosis apparatus 100, such as a walking speed and a frailty index.

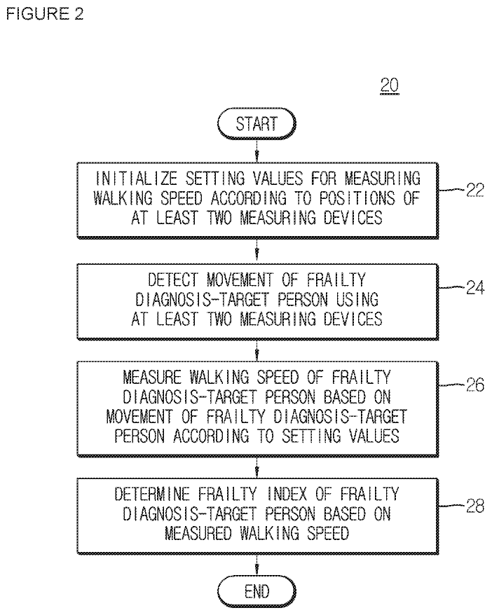

FIG. 2 illustrates an embodiment of a frailty diagnosis method 20 of determining the frailty index of a frailty diagnosis-target person based on the walking speed of the frailty diagnosis-target person.

In operation 22, setting values for measuring the speed of walking according to the positions of at least two measuring devices are set by a processor. The relative distance between the measuring devices may be calculated according to the positions of the measuring devices. To reflect changes in the positions of the measuring devices, setting of the setting values may be performed when the measuring devices are moved, or periodically according to a predetermined measurement period.

In operation 24, the at least two measuring devices detect the movement of a frailty diagnosis-target person. The measuring devices may transmit the detection times at which the frailty diagnosis-target person was detected to a main body of a frailty diagnosis apparatus. Information about the detection times may be transmitted through an encoding and modulation process. As the detection times at which the frailty diagnosis-target person was detected are transmitted from the at least two measuring devices to the main body of the frailty diagnosis apparatus, information necessary for calculating the speed of walking of the frailty diagnosis-target person is prepared.

In operation 26, the processor acquires the speed of walking of the frailty diagnosis-target person based on the movement of the frailty diagnosis-target person according to the setting values. The speed of walking is calculated based on the detection times detected by the measuring devices in operation 24 and the setting values determined with respect to the relative distance between the measuring devices in operation 22.

In operation 28, the processor determines the frailty index of the frailty diagnosis-target person based on the speed of walking measured in operation 26. In an embodiment, a walking speed parameter representing a walking speed in a specific section may be determined, and the frailty index may be determined according to the walking speed parameter. In another embodiment, the frailty index may be determined based on the speed of walking with reference to a walking speed-frailty index correlation indicating a relationship between walking speed and frailty index. In another embodiment, the frailty index may be determined by considering not only the speed of walking but also other measured values related to the frailty index.

The frailty diagnosis method 20 may be performed using the frailty diagnosis apparatus 100 illustrated in FIG. 1. Therefore, the functions of the elements of the frailty diagnosis apparatus 100 may be additionally used in the frailty diagnosis method 20.

FIGS. 3A and 3B are two-dimensional graphs respectively illustrating a relationship between chronological age and walking speed and a relationship between frailty index and walking speed. The graphs of FIGS. 3A and 3B were obtained through linear regression analysis of statistical data, and the relationship between chronological age and walking speed and the relationship between frailty index and walking speed are shown in the form of a linear function in FIGS. 3A and 3B.

The x-axis in FIG. 3A refers to an average walking speed in m/s. In addition, the y-axis in FIG. 3A refers to actual age. Referring to FIG. 3A, it may be seen that the average walking speed decreases as the actual age increases, Therefore, it may be seen that the walking speed has a correlation with frailty.

The x-axis in FIG. 3B refers to an average walking speed in m/s. In addition, the y-axis in FIG. 3B refers to a frailty index, Referring to FIG. 3B, it may be seen that the average walking speed decreases as the frailty index increases. Therefore, it may be seen that the walking speed has a correlation with frailty.

Therefore, referring to FIGS. 3A and 3B, the physiological age and frailty index of a frailty diagnosis-target person may be estimated by measuring the walking speed of the frailty diagnosis-target person.

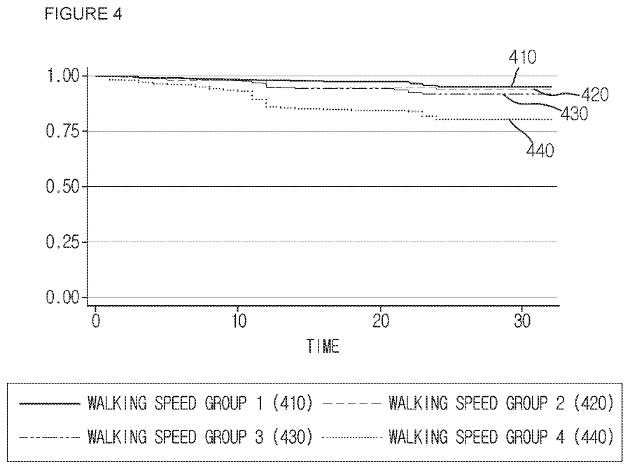

FIG. 4 is a graph illustrating the survival probabilities of groups having certain walking speeds by Kaplan-Meier analysis. Kaplan-Meier analysis concerns the probability of survival over time of people having specific conditions, which may be obtained by observing a sufficiently large sample population for a long period of time.

The x-axis in FIG. 4 refers to the proportion of survivors to the total cohort participants, and the y-axis in FIG. 4 refers to a measurement period Referring to FIG. 4, the cohort participants are classified into four groups 410, 420, 430, and 440 according to the walking speeds thereof, and results of observation of deaths over the measurement period are shown for each group. The proportion of survivors in the group 440 having the lowest walking speed has decreased the most, and the proportion of survivors in the group 410 having the highest walking speed has decreased the least. That is, the higher walking speed the group has, the higher survival rate the group has.

Therefore, the frailty diagnosis apparatus 100 may estimate the survival probability of a frailty diagnosis-target person by measuring the walking speed of the frailty diagnosis-target person.

Table 1 below shows items strongly related to the speed of walking. When a population group is divided into participants (high speed walkers) who walk faster than the median speed and participants (low speed walkers) who walk slower than the median speed, statistically significant differences are observed therebetween in multimorbidity, grip strength, short physical performance battery (SPPB), frailty indexes (K-FRAIL and CHS frailty score), activities of daily living and instrumental activities of daily living (ADL, IADL), depression, cognition, polypharmacy, fall history, etc. That is, it is possible to infer the health state of a frailty diagnosis-target person by measuring the walking speed of the frailty diagnosis-target person.

TABLE 1

| | Low speed | High speed | |
|---|---|---|---|
| Items | walkers | walkers | P value |
| multimorbidity (n) | 310.00 | 221.00 | <0.001 |
| Dominant grip strength | 19.92 | 24.90 | <0.001 |
| (mean, sd) | | | |
| SPPB score (mean, sd) | 6.61 | 9.37 | <0.001 |
| K-FRAIL score (mean, sd) | 1.63 | 0.93 | <0.001 |
| CHS score (mean, sd) | 2.39 | 1.25 | <0.001 |
| ADL disability (n, %) | 125.00 | 56.00 | <0.001 |
| IADL disability (n, %) | 294.00 | 150.00 | <0.001 |
| Depression (n, %) | 102.00 | 34.00 | <0.001 |
| Cognitive dysfunction (n, %) | 270.00 | 125.00 | <0.001 |
| Polypharmacy (n, %) | 193.00 | 113.00 | <0.001 |
| Fall history for previous | 0.33 | 0.16 | 0.001 |
| 1 year (mean, sd) | | | |

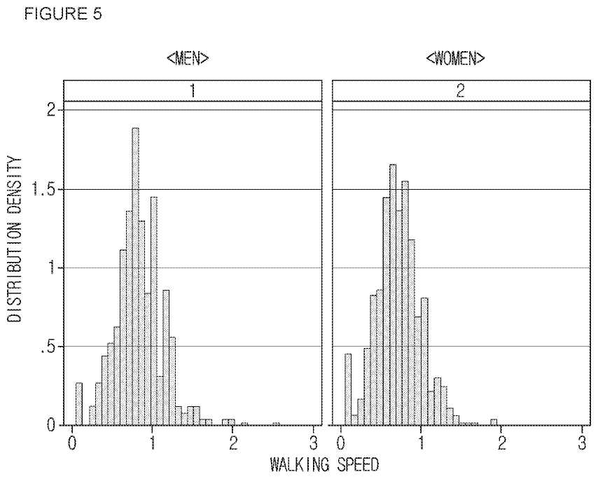

FIG. 5 illustrates distributions of the walking speed of older adults living in a community in Korea according to the genders. The left graph in FIG. 5 shows a distribution of the walking speed of men. The right graph in FIG. 5 shows a distribution of the walking speed of women. Since there is a difference in the distribution of walking speed between men and women, it is necessary to consider the gender of a frailty diagnosis-target person in addition to the walking speed of the frailty diagnosis-target person when measuring the physiological age of the frailty diagnosis-target person.

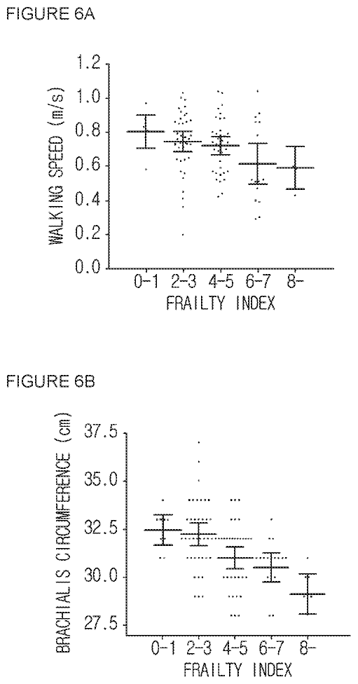

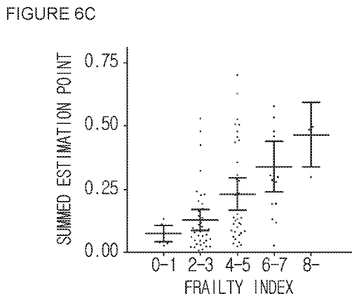

FIGS. 6A to 6C are graphs for estimating a frailty index by using a walking speed and other measured values, Referring to FIGS. 6A to 60, the speed of walking and the circumference of brachialis are measured to show a correlation between the frailty index and the sum of a walking speed parameter and a brachialis circumference parameter. The brachialis circumference parameter is related to the muscle function of a frailty diagnosis-target person and is thus closely related to the frailty index of the frailty diagnosis-target person. Thus, the brachialis circumference parameter is an important factor together with the speed of walking when estimating a frailty index.

The graph of FIG. 6A shows a correlation between the walking speed parameter and the frailty index. Referring to the graph of FIG. 6A, as the walking speed parameter decreases, the frailty index increases.

The graph of FIG. 6B shows a correlation between the brachialis circumference parameter and the frailty index. Referring to the graph of FIG. 6B, as the brachialis circumference parameter decreases, the frailty index increases.

The graph of FIG. 6C shows a correlation between the frailty index and an evaluation value obtained based on the walking speed parameter and the brachialis circumference parameter according to the results shown in the graphs of FIGS. 6A and 6B. Referring to the graph of FIG. 6C, as the evaluation value increases, the frailty index increases. The accuracy of estimation of a frailty index may increase by considering two or more factors in combination. In FIGS. 6A to 6C, the evaluation value obtained by combining the walking speed and the brachial circumference is used. However, another factor may be used instead of or in addition to the brachialis circumference to estimate the frailty index.

Embodiments have been described above in detail with reference to the accompanying drawings so that those of ordinary skill in the art may easily implement the present disclosure. In the drawings, portions not related to the present disclosure are omitted for clarity of description.

The above-described embodiments may be written as computer-executable programs and may be implemented in general-purpose digital computers that execute the programs using a non-transitory computer-readable recording medium.

The terms used in the present specification are selected based on general terms currently widely used in the art in consideration of functions regarding the present disclosure, but the terms may vary according to the intention of those of ordinary skill in the art, precedents, or new technology in the art. Also, some terms may be arbitrarily selected by the applicants, and in this case, the meaning of the selected terms are described in the detailed description of the present disclosure. Thus, the terms used herein should not be construed based on only the names of the terms but should be construed based on the meaning of the terms together with the description throughout the present disclosure.

The terms of a singular form may include plural forms unless referred to the contrary. In the present specification, when it is described that a part or portion “includes” and/or “comprises” a particular element, the presence or addition of one or more other elements in the part or portion is not precluded, and the part or portion may include or comprise one or more other elements unless otherwise specified.

While some best embodiments of the present disclosure have been described, it will be apparent to those of ordinary skill in the art that substitutions, modifications, and changes may be made therefrom. That is, the claims may include all of such substitutions, modifications, and changes. Therefore, all described in the present specification including the drawings should be construed in an illustrative and non-limiting sense.

1. A frailty diagnosis method comprising:
detecting, by a measuring devices, a movement of a frailty diagnosis-target person;
measuring, by a processor, a walking speed of the frailty diagnosis-target person based on the movement of the frailty diagnosis-target person;
determining, by the processor, a walking speed parameter representing the measured walking speed based on the measured walking speed; and
predicting, by the processor, a frailty index of the frailty diagnosis-target person based on the walking speed parameter.

2.-6. (canceled)

7. The frailty diagnosis method of claim 1, wherein the walking speed parameter represents a walking speed in a specific section.

8. The frailty diagnosis method of claim 1, wherein the determining of the frailty index of the frailty diagnosis-target person is performed by
determining the frailty index based on the walking speed according to a walking speed-frailty index correlation function, which indicates a relationship between the walking speed and the frailty index.

9. The frailty diagnosis method of claim 1, wherein the determining of the frailty index of the frailty diagnosis-target person comprises:
obtaining frailty parameters of the frailty diagnosis-target person in addition to a walking speed parameter of the frailty diagnosis-target person; and
determining the frailty index of the frailty diagnosis-target person based on the walking speed parameter and at least one of the frailty parameters,
wherein the frailty parameters comprise a muscle decrease parameter relating to a muscle function of the frailty diagnosis-target person and a motor ability parameter relating to motor ability of the frailty diagnosis-target person.

10. The frailty diagnosis method of claim 1, further comprising inferring, by the processor, a health state of the frailty diagnosis-target person based on the frailty index, age, and gender of the frailty diagnosis-target person.

11. The frailty diagnosis method of claim 1, further comprising displaying, on a display, at least one of the walking speed and the frailty index.

12. A frailty diagnosis apparatus comprising:
a measuring devices configured to detect a movement of a frailty diagnosis-target person; and
a processor configured to measure a walking speed of the frailty diagnosis-target person based on the movement of the frailty diagnosis-target person, determine a walking speed parameter representing the measured walking speed based on the measured walking speed, and predict a frailty index of the frailty diagnosis-target person based on the walking speed parameter.

13. A non-transitory computer-system-readable storage medium having recorded thereon a program for executing the frailty diagnosis method of claim 1.

14. The frailty diagnosis method of claim 1, wherein the frailty index indicates a ratio of a number of frailty-related symptoms that the frailty diagnosis-target person has to a total number of frailty-related symptoms.

15. The frailty diagnosis method of claim 1, further comprising setting, by the processor, a setting value for measuring a walking speed in a given measuring environment according to positions of the measuring device.

16. The frailty diagnosis method of claim 15, wherein, when at least two measuring devices are used, the setting of the setting value is performed according to relative positions of the at least two measuring devices.

17. The frailty diagnosis method of claim 15, wherein the setting of the setting value is periodically performed according to a preset period.

18. The frailty diagnosis method of claim 15, wherein the setting of the setting value is performed when a variation in the positions of the measuring devices is detected.

19. A computer program product for executing the frailty diagnosis method of claim 1.
